(19) United States
(12) Patent Application Publication
(10) Pub. No.: US 2002/0178490 A1
(43) Pub. Date: Dec. 5, 2002

(54) Underwater mask with protective covering integral to the headrest fit to hold and envelop the mask itself

(76) Inventor: Andrea Silvestri, Genova (IT)

Correspondence Address: LARSON & TAYLOR, PLC, 1199 NORTH FAIRFAX STREET, SUITE 900, ALEXANDRIA, VA, 22314, US

(21) Appl. No.: 09/988,632

(22) Filed: Nov. 20, 2001

(30) Foreign Application Priority Data
May 30, 2001 (IT) GE2001 U 000015

(51) Int. Cl. G02C 1/00; A61F 9/02

(52) U.S. Cl. 2/428; 351/43

(57) ABSTRACT

Underwater mask including a protective covering integral to the headrest fit to envelop and hold the mask itself. To this aim the headrest (4) of the mask is made in the shape of an envelope element provided with a foldable band, fit to hold and envelop the mask within in the manner of a bag. Said envelope element is connected to the mask belts so to form the headrest of the mask itself, and it is made of rubber or rubber like elastomeric material.

### TITLE OF THE INVENTION

Underwater mask with protective covering integral to the headrest fit to hold and envelop the mask itself

### BACKGROUND AND SUMMARY OF THE INVENTION

The present invention refers to the underwater masks, and more in particular it refers to the protection coverings of said masks.

It is well-known that the glasses of the underwater masks, an in particular the glasses of the so said “optical” ones where the mask glass is optically treated in order to compensate for user's sight defects, are particularly fragile and subjected to be scored or even splintered during the carrying in the bags where masks are generally put together with all the diver's equipment.

The same owner of the present patent application has proposed in the past shock resistant cases for the protection of the glasses of the underwater masks, which have been object of the Italian patent for utility model No. 231.846. These cases, even though they performed completely the task they were invented for, present however the disadvantage that, being completely independent from the mask, they are often left behind, or they are not always within reach of the diver, then he often puts down the mask without any protection together with the other equipment.

Then the aim of the present invention is to obviate these disadvantages of the known underwater masks cases, by embodying a case which is integral with the headrest of the mask itself, so that the case is not materially dissociable from the mask.

Said aim is obtained by embodying the mask headrest in the shape of a foldable envelope element, which when unwound and unfolded can envelop the mask, holding it within in the manner of a bag. Advantageously said element is made integral with the mask belts, and it is made of rubber or rubber like elastomeric material.

### BRIEF DESCRIPTION OF THE DRAWINGS

Further aims and advantages of the present invention will be better cleared during the following description of a preferred embodiment of the same, drawn with reference to the enclosed drawings, where:

FIG. 1 is a perspective rear view, this is to say from the headrest side, of an underwater mask according to the present invention;

FIG. 2 is a side elevation view of the mask of FIG. 1, with the headrest longitudinally sectioned, and

FIG. 3 shows the headrest unwound and expanded which envelops the mask, holding it within in the manner of a bag.

### DESCRIPTION OF THE PREFERRED EMBODIMENT OF THE INVENTION

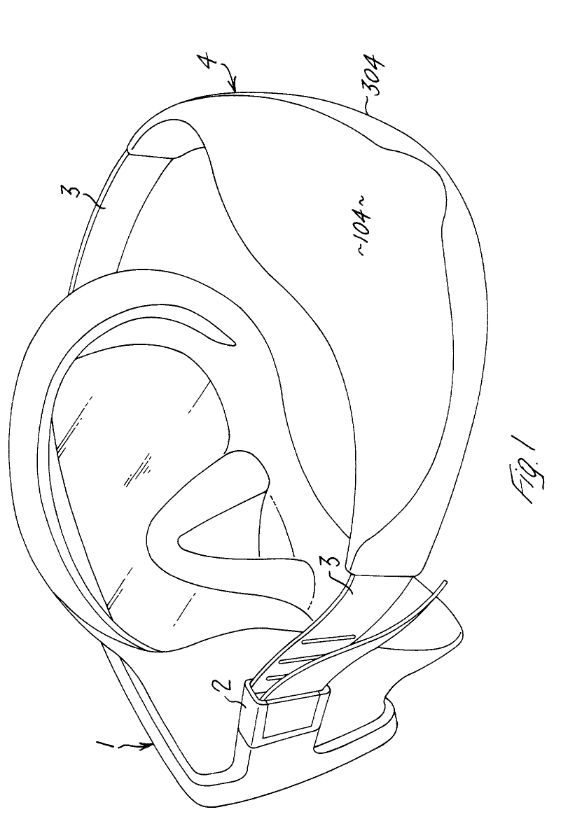

With reference to the drawings, and with particular reference to FIG. 1 of them, with 1 is generically indicated a mask for underwater activities. Said mask includes, in a well-known way in itself, two side buckles 2 to which one end of the two belts 3 is connected. The other ends of said belts being in turn connected to the two ends of the headrest 4.

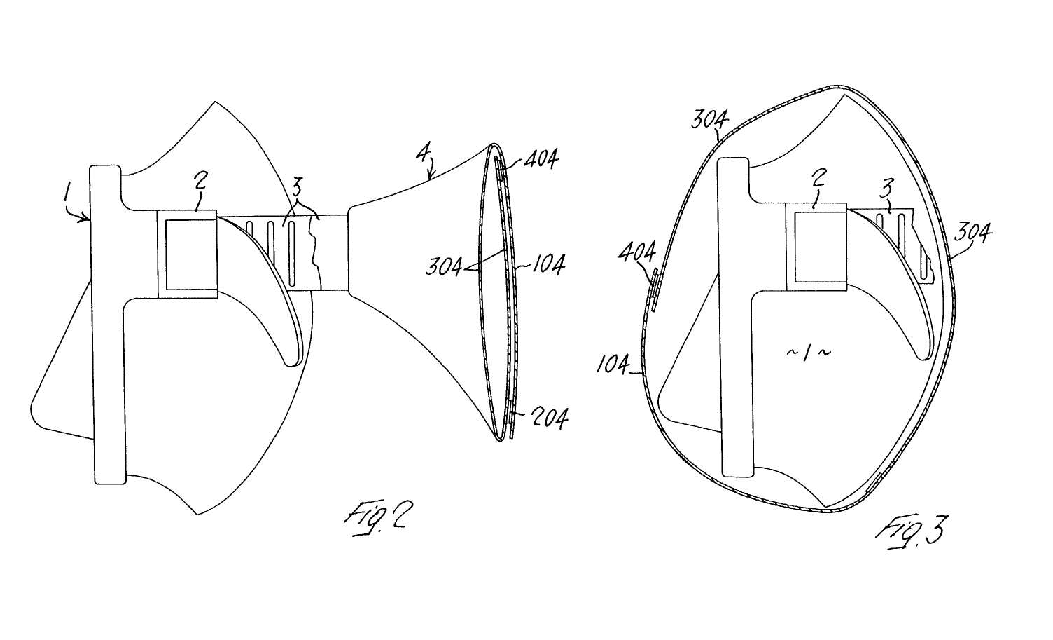

Said headrest 4 is made, as better shown in FIG. 2, in the shape of an envelope with closing band 104 locked in the edge by a closing element 204, such as for instance a Velcro closing element, or the like. With the headrest folded on itself, as shown in FIGS. 1 and 2, the mask can be worn in the usual way, and thanks to the large headrest 4 its application on the nape proves to be quite comfortable and moreover it is avoided the sliding of the belt along the nape, which the normal belts of the underwater masks are often subjected to.

When it is desired to put down the mask 1, this is made in the following way: the closing element 204 is released and the band 104 is raised, then the bag 304 is folded so that its inner side happens to be on the outside, and vice versa, and the mask is inserted, with the belts 3, into the so obtained folded bag, then the enveloping of the mask is completed by securing the band 104 through a Velcro closing 404. In this way, the mask 1 will prove to be completely held within the new bag 304-104, which has been extended around it. To bring the mask back to use mode, the operations described are repeated in the opposite way.

It is then clear that, thanks to the described embodiment, the covering will always accompany the mask itself.

I claim:

1. An underwater mask comprising a mask body; means at the two sides of said body for the connection of one end of two side belts, the other end of the said two side belts being in turn connected to the two opposite ends of a headrest, and further comprising a protective bag integral to the said headrest fit to envelop and hold the mask body with the said side belts and the said headrest.

2. An underwater mask according to claim 1, in which the said headrest is made in the shape of a bag element provided with a foldable band fit to hold and envelop the said mask within in the manner of a bag.

2) An underwater mask according to claim 2, in which said bag element is connected with, or is made integral with the said mask belts so to form the said headrest of the said mask itself.

3) An underwater mask according to claim 3, in which said bag element is made of rubber or rubber like elastomeric material.

4. An underwater mask according to claim 1 in which said bag element is provided with a closing foldable band locked at its edge by a suitable closing element.
